(19) United States
(12) Patent Application Publication
(10) Pub. No.: US 2022/0146658 A1
(43) Pub. Date: May 12, 2022

(54) BIOLOGICAL DETECTION DEVICE

(71) Applicant: AISIN CORPORATION, Kariya (JP)

(72) Inventor: Takashi OTANI, Kariya-shi (JP)

(73) Assignee: AISIN CORPORATION, Kariya (JP)

(21) Appl. No.: 17/452,590

(22) Filed: Oct. 28, 2021

(30) Foreign Application Priority Data
Nov. 9, 2020 (JP) 2020-186797

(51) Int. Cl. G01S 13/56; G01S 7/41; G01S 13/89; A61B 5/00

(52) U.S. Cl. CPC G01S 13/56; A61B 5/6893; G01S 13/89; G01S 7/415

(57) ABSTRACT

A biological detection device includes: a transmission unit transmitting a transmission wave into a compartment; a reception unit receiving a reflected wave; a creation unit creating three-dimensional map information on the compartment based on the reflected wave; a specifying unit specifying a position of a reflection source, which is an object having a reflection intensity equal to or greater than a predetermined value, in the three-dimensional map information when the vehicle where a person is seated is stopped; an object detection unit detecting a motion of an object in the compartment based on a Doppler shift of the reflected wave and outputting a detection result; and a person detection unit determining that there is no person at a position at which no person is detected in an immediately preceding detection cycle and which substantially matches the position of the reflection source.

### CROSS REFERENCE TO RELATED APPLICATIONS

This application is based on and claims priority under 35 U.S.C. § 119 to Japanese Patent Application 2020-186797, filed on Nov. 9, 2020, the entire content of which is incorporated herein by reference.

### TECHNICAL FIELD

This disclosure relates to a biological detection device.

### BACKGROUND DISCUSSION

In the related art, there is a technique of calculating biological information (pulse wave, respiration, body motion, and the like) by irradiating a person with radio waves and performing frequency analysis on a Doppler shift of reflected waves. There is also a technique of determining presence or absence of a person (occupant) in a passenger compartment of a vehicle (passenger car or the like) based on transmission and reception of radio waves of a frequency modulated continuous wave (FMCW) system. In the FMCW system, a position irradiated with the radio waves and where reflected waves are reflected and a velocity at the position can be obtained from the Doppler shift. When these techniques are combined, for example, it is considered that the person in the passenger compartment or biological information thereof can be detected with high accuracy when the vehicle is stopped. For example, see JP2019-126407A (which will be referred to also as Patent reference 1) and JP2020-101415A (which will be referred to also as Patent reference 2).

However, in the related art, it is considered that when the vehicle travels, components and the person in the passenger compartment also vibrate due to vibration of the vehicle, and detection accuracy of the person decreases.

A need thus exists for a biological detection device which is not susceptible to the drawback mentioned above.

### SUMMARY

A biological detection device according to an embodiment includes: a transmission unit configured to transmit a FMCW modulated transmission wave into a passenger compartment of a vehicle; a reception unit configured to receive a reflected wave generated by the transmission wave being reflected by an object in the passenger compartment; a creation unit configured to create three-dimensional map information on the passenger compartment based on the reflected wave; a specifying unit configured to specify a position of a reflection source, the reflection source being an object having a reflection intensity equal to or greater than a predetermined value with respect to the transmission wave, in the created three-dimensional map information when the vehicle in which at least one person is seated in a seat in the passenger compartment is stopped; an object detection unit configured to detect a motion of an object in the passenger compartment based on a Doppler shift of the reflected wave and output a detection result; and a person detection unit configured to detect, when the vehicle is stopped, a person based on the three-dimensional map information and the detection result in a predetermined detection cycle, and detect, when the vehicle is traveling, a person based on the three-dimensional map information and the detection result in a predetermined detection cycle, and determine that there is no person at a position at which no person is detected in an immediately preceding detection cycle and which substantially matches the position of the reflection source among positions at which persons are detected when the number of detected persons increases compared to the number of detected persons when the vehicle is stopped.

### BRIEF DESCRIPTION OF THE DRAWINGS

The foregoing and additional features and characteristics of this disclosure will become more apparent from the following detailed description considered with the reference to the accompanying drawings, wherein:

FIG. 1 is a schematic diagram of a vehicle according to an embodiment;

FIG. 2 is a block diagram showing functional configurations of a radio wave sensor and a controller according to the embodiment;

FIGS. 3A-3C are diagrams showing an outline of signal processing by a FMCW method according to the embodiment;

FIG. 4 is a diagram schematically showing a difference between a detection result when the vehicle is stopped and a detection result when the vehicle is traveling according to the embodiment;

FIG. 5 is a flowchart showing processing performed by the controller according to the embodiment;

FIG. 6 is a flowchart showing details of processing of step S10 in FIG. 5;

FIG. 7 is a schematic diagram of a vehicle according to a modification; and

FIG. 8 is a schematic diagram of a vehicle according to a modification.

### DETAILED DESCRIPTION

Hereinafter, an embodiment of a biological detection device disclosed here will be described with reference to the drawings.

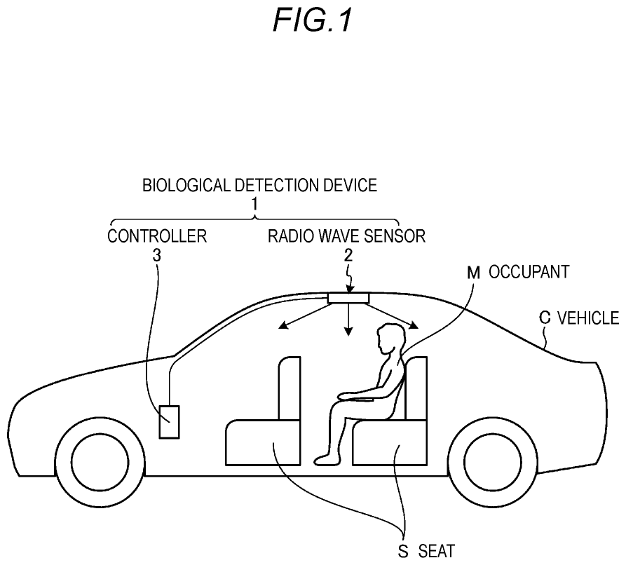

FIG. 1 is a schematic diagram of a vehicle C according to the embodiment. A radio wave sensor 2 and a controller 3 constituting a biological detection device 1 are disposed in a passenger compartment of the vehicle C. The radio wave sensor 2 is disposed at a ceiling portion in the passenger compartment. The controller 3 is disposed, for example, in a dashboard provided at a front end portion of the passenger compartment.

An occupant M is seated in a seat S. In the following description, presence of the occupant M (person) is detected by the biological detection device 1, and biological information (pulse wave, respiration, body motion, and the like) on the occupant M is calculated. In the following description, among objects in the passenger compartment, an object having a reflection intensity equal to or greater than a predetermined intensity with respect to a transmission wave emitted by a transmission unit 21 of the radio wave sensor 2 is referred to as a reflection source. The reflection source is mainly a metal.

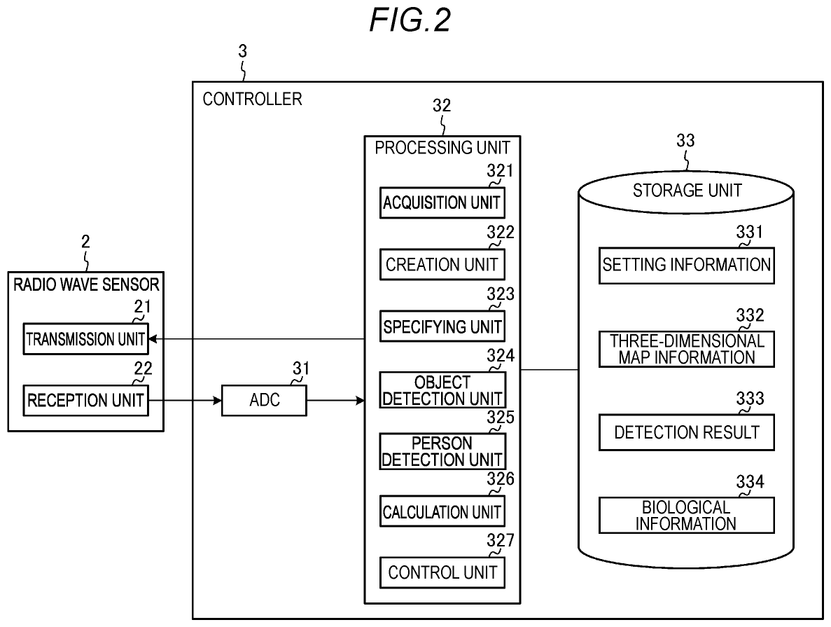

FIG. 2 is a block diagram showing functional configurations of the radio wave sensor 2 and the controller 3 according to the embodiment. The radio wave sensor 2 includes the transmission unit 21 and a reception unit 22.

The transmission unit 21 transmits (radiates) a FMCW modulated transmission wave in a wide range into the passenger compartment of the vehicle C. The reception unit 22 receives a reflected wave generated by the transmission wave being reflected by an object in the passenger compartment. The reception unit 22 includes plural receiving antennas.

The controller 3 is constituted by, for example, a micro controller unit (MCU) having an integrated circuit on which a hardware processor, a memory, and the like are mounted. The controller 3 includes an analog-to-digital converter (ADC) 31, a processing unit 32, and a storage unit 33.

The ADC 31 converts an analog signal acquired from the reception unit 22 of the radio wave sensor 2 into a digital signal and outputs the digital signal to the processing unit 32.

The storage unit 33 is a storage device such as a random access memory (RAM), a read only memory (ROM), a solid state drive (SSD), or a hard disk drive (HDD). The storage unit 33 stores a program executed by the processing unit 32, data necessary for executing the program, data generated by executing the program, and the like. The storage unit 33 stores, for example, setting information 331, three-dimensional map information 332, a detection result 333, and biological information 334.

The setting information 331 stores various kinds of setting information such as a frequency range for determining a biological signal of a person and various kinds of threshold values (a reflection source threshold value for specifying a reflection source, a person threshold value for detecting a person, and the like).

The three-dimensional map information 332 is information indicating a three-dimensional object arrangement state in the passenger compartment, and is created by a creation unit 322 based on reflected wave information.

The detection result 333 is information on a detection result by an object detection unit 324.

The biological information 334 is biological information on a person calculated by a calculation unit 326.

The processing unit 32 is constituted by, for example, a hardware processor such as a central processing unit (CPU). The processing unit 32 reads the program stored in the storage unit 33 and executes arithmetic processing. The processing unit 32 includes, as functional units, an acquisition unit 321, the creation unit 322, a specifying unit 323, the object detection unit 324, a person detection unit 325, the calculation unit 326, and a control unit 327. Some or all of the units 321 to 327 may be constituted by hardware such as a circuit including an application specific integrated circuit (ASIC) or a field-programmable gate array (FPGA).

The acquisition unit 321 acquires the reflected wave information from the ADC 31.

The creation unit 322 creates three-dimensional map information on the passenger compartment based on the reflected wave information, and stores the three-dimensional map information as the three-dimensional map information 332 in the storage unit 33.

The specifying unit 323 specifies a position of the reflection source in the three-dimensional map information 332 created when the vehicle C is stopped.

The object detection unit 324 detects a motion of an object in the passenger compartment based on a Doppler shift of the reflected wave, and stores a detection result in the storage unit 33 as the detection result 333.

The person detection unit 325 detects the occupant M based on the setting information 331, the three-dimensional map information 332, and the detection result 333 at a predetermined detection cycle when the vehicle C is stopped. Further, the person detection unit 325 detects the occupant M based on the setting information 331, the three-dimensional map information 332, and the detection result 333 when the vehicle C is traveling in the predetermined detection cycle, and determines that there is no occupant M at a position at which no occupant M is detected in an immediately preceding detection cycle and which substantially matches a position of the reflection source (including a case of an exact match and a case of a close match) among positions at which occupants M are detected when the number of detected occupants M increases compared to the number of detected occupants M during stop.

The calculation unit 326 calculates the biological information with a motion related to the occupant M based on the detection result 333 and the like. An example of the biological information is a heart rate of the occupant M. When the transmission wave from the transmission unit 21 is reflected by a chest of the occupant M, an influence of a Doppler effect by the chest vibrating in a front-rear direction is reflected in the reflected wave. Therefore, the calculation unit 326 calculates the biological information based on a Doppler frequency derived from a signal of the reflected wave.

The calculation unit 326 calculates the biological information on the occupant M based on the detection result 333 and the like when the vehicle C is traveling, and removes a component derived from vibration of the vehicle C from the biological information based on a motion of the reflection source that is close to the seat S in which the occupant M is seated and vibrates in correlation with the vibration when the seat S vibrates.

The control unit 327 executes an operation other than operations performed by the units 321 to 326.

Processing of the units 324 to 326 can be more specifically performed, for example, as follows. According to a propagation velocity and an amplitude of a biological signal (person signal), the following three types of analysis methods (1) to (3) are simultaneously performed. A case where the propagation velocity of the biological signal can be detected by the Doppler shift is referred to as a large velocity, and a case where the propagation velocity is a velocity at which sufficient detection accuracy cannot be obtained is referred to as a small velocity. The magnitude of the amplitude of the biological signal that can be detected by a 3D (Dimensions) voxel is defined as a large amplitude, and an amplitude is defined as a small amplitude when the sufficient detection accuracy cannot be obtained.

### (1) Extraction of Biological Signal Having Large Velocity and Large Amplitude

Plural reflection sources correlated with vibration of the seat S are determined in advance, and the vibration of the seat S and vibration transmitted from the seat S to the occupant M are predicted from vibration of the reflection sources and the number, positions, and vibration of persons in the seat S. For example, data such as the vibration of the seat S alone and vibration when the occupant M is present is acquired in advance by an experiment, and a correlation therebetween is specified in advance to perform prediction. For example, noise having the same frequency as the biological signal is removed by subtracting a velocity distribution having the same frequency as the biological signal among the predicted vibration of the vehicle C from a velocity distribution of the occupant M obtained from the Doppler shift.

### (2) Extraction of Biological Signal Having Large Velocity and Small Amplitude

Since the amplitude of the biological signal is sufficiently small, it may be considered that the biological signal is not included in vibration of the 3D voxel. Therefore, a velocity distribution having the same frequency as the biological signal is extracted from the velocity distribution of a 3D voxel signal derived from a person and is subtracted from a velocity distribution of a person obtained from the Doppler shift to remove noise having the same frequency as the biological signal.

### (3) Extraction of Biological Signal Having Small Velocity and Large Amplitude

The plural reflection sources correlated with the vibration of the seat S are determined in advance, and the vibration of the seat S and the vibration transmitted from the seat S to a person are predicted from vibration of the reflection sources and the number, positions, and vibration of persons in the seat S. For example, the data such as the vibration of the seat S alone and the vibration when the occupant M is present is acquired in advance by the experiment, and the correlation therebetween is specified in advance to perform prediction. For example, noise derived from the vibration of the vehicle C is removed from the biological information by subtracting the vibration transmitted from the vehicle C to the person from the 3D voxel signal derived from the person.

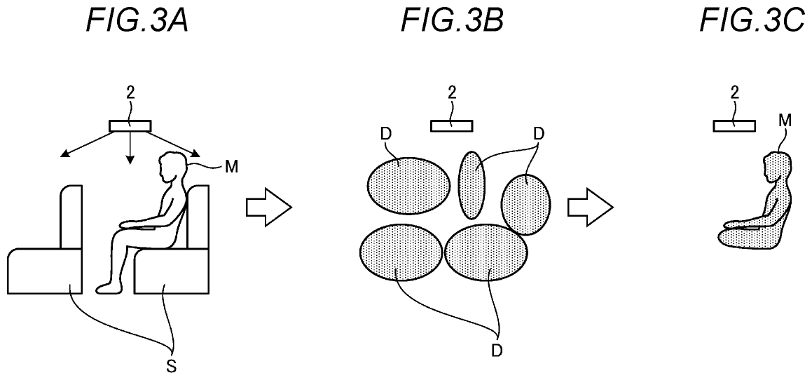

FIGS. 3A-3C are diagrams showing an outline of signal processing by a FMCW method according to the embodiment. As shown in FIG. 3A, first, the FMCW modulated transmission wave is transmitted from the transmission unit 21 of the radio wave sensor 2 into the passenger compartment of the vehicle C. The reception unit 22 of the radio wave sensor 2 receives the reflected wave.

Next, as shown in FIG. 3B, the creation unit 322 creates the three-dimensional map information in the passenger compartment based on the reflected wave. The three-dimensional map information includes, in addition to the occupant M, information on an object (reflection source) such as a metal having a high reflectance. That is, it is not possible to distinguish whether a recognized object is a metal or a person by using the three-dimensional map information alone.

Next, as shown in FIG. 3C, the occupant M is detected by object detection based on the Doppler shift of the reflected wave by the object detection unit 324 and person detection by the person detection unit 325. The biological information on the occupant M can also be calculated by the calculation unit 326.

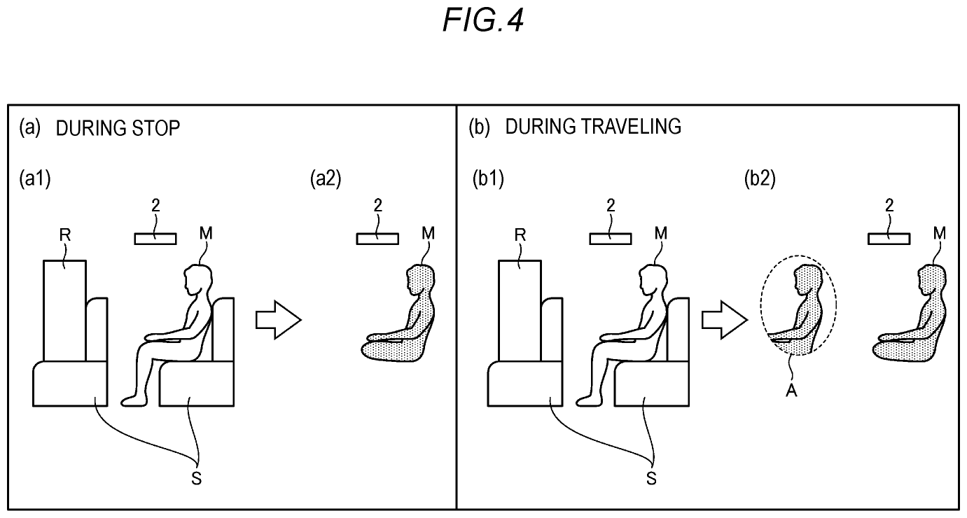

FIG. 4 is a diagram schematically showing a difference between a detection result when the vehicle is stopped and a detection result when the vehicle is traveling according to the embodiment. First, detection during stop will be described with reference to (a) of FIG. 4. (a1) of FIG. 4 shows an actual state of the passenger compartment, and (a2) of FIG. 4 shows the detected occupant M.

The creation unit 322 creates a 3D signal before extracting a signal of a person. The creation unit 322 determines a reflection source threshold value based on a maximum value of an intensity of the signal or the like. For example, a larger value of a value of tens of percent of the maximum value of the intensity and an experimental value is determined as the reflection source threshold value.

Next, the specifying unit 323 specifies the position of the reflection source in the three-dimensional map information 332 using the reflection source threshold value.

Next, the object detection unit 324 detects the motion of the object in the passenger compartment based on the Doppler shift of the reflected wave.

Next, the person detection unit 325 extracts a person signal by frequency analysis. The person detection unit 325 determines a person threshold value based on a maximum value of an intensity of the person signal or the like. For example, a larger value of a value of tens of percent of the maximum value of the intensity and an experimental value is determined as the person threshold value. The person detection unit 325 detects one or more occupants M and labels person signals corresponding to the occupants M in descending order of intensity.

When a reflection source R made of metal or the like is present in the passenger compartment, the reflection source R is in a stationary state when the vehicle is stopped, and thus the person detection unit 325 does not erroneously detect the reflection source R as a person.

Next, the calculation unit 326 performs frequency analysis on each of the labeled person signals and calculates biological information. In this case, for example, when a component due to vibration derived from an object other than a person is mixed in the person signal after an engine is started or due to strong winds or the like, the component due to the vibration derived from the object other than the person is removed in the same manner as in a case of detection during traveling, which will be described later.

Next, detection during traveling will be described with reference to (b) of FIG. 4. (b1) of FIG. 4 shows an actual state of the passenger compartment, and (b2) of FIG. 4 shows the detected occupant M and an erroneous detection result (region A).

The creation unit 322 creates a 3D signal before extracting a signal of a person. The creation unit 322 determines a reflection source threshold value based on a maximum value of an intensity of the signal or the like. For example, a larger value of a value of tens of percent of the maximum value of the intensity and an experimental value is determined as the reflection source threshold value.

Next, the specifying unit 323 specifies the position of the reflection source in the three-dimensional map information 332 using the reflection source threshold value.

Next, the object detection unit 324 detects the motion of the object in the passenger compartment based on the Doppler shift of the reflected wave.

Next, the person detection unit 325 extracts a person signal by frequency analysis. The person detection unit 325 determines a person threshold value based on a maximum value of an intensity of the person signal or the like. For example, a larger value of a value of tens of percent of the maximum value of the intensity and an experimental value is determined as the person threshold value. The person detection unit 325 labels person signals in descending order of intensity.

When the reflection source R made of metal or the like is present in the passenger compartment, the reflection source R may vibrate during traveling, and thus the person detection unit 325 may erroneously detect the reflection source R as a person. Therefore, erroneous detection is prevented in the following manner.

The person detection unit 325 detects the occupant M, and determines that there is no occupant M at the position at which no occupant M is detected at the immediately preceding detection cycle and which substantially matches the position of the reflection source R among positions at which the occupants M are detected when the number of the detected occupants M increases compared to the number of the detected occupants M during stop. Accordingly, it is possible to prevent erroneous detection of a person. When a person signal that is not actually a person is excluded, re-labeling is performed thereafter.

Next, the calculation unit 326 performs the frequency analysis on each of the labeled person signals and calculates the biological information. In this case, the calculation unit 326 removes the component derived from the vibration of the vehicle C from the biological information based on the motion of the reflection source that is close to the seat S in which the occupant M is seated and vibrates in correlation with the vibration when the seat S vibrates. Regarding this correlation, for example, in consideration of the number, positions, sizes, and the like of the occupants M, a reflection source that vibrates in correlation with the vibration of the seat S may be specified in advance by an experiment, or data on a relation between the vibration of the seat S and the vibration of the reflection source in this case may be created and used.

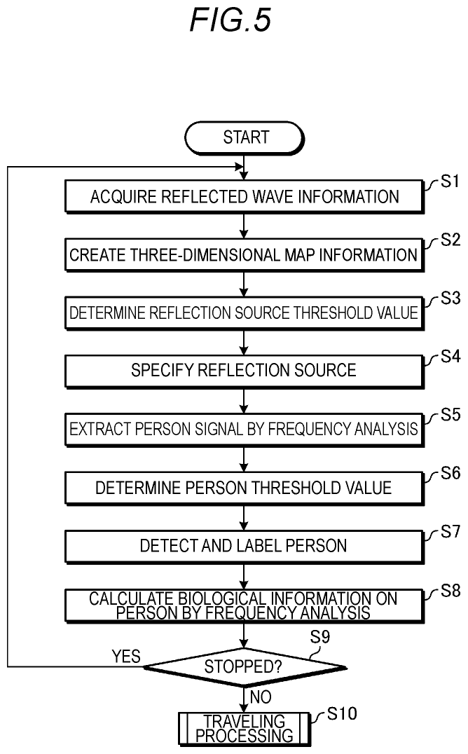

FIG. 5 is a flowchart showing processing performed by the controller 3 according to the embodiment. In step S1, the acquisition unit 321 acquires reflected wave information from the ADC 31.

Next, in step S2, the creation unit 322 creates the three-dimensional map information 322 on the passenger compartment based on the reflected wave information.

Next, in step S3, the creation unit 322 determines a reflection source threshold value based on a maximum value of an intensity of a signal or the like.

Next, in step S4, the specifying unit 323 specifies a position of a reflection source in the three-dimensional map information 332 using the reflection source threshold value.

Next, in step S5, after the object detection unit 324 detects an object in the passenger compartment, the person detection unit 325 extracts a person signal by frequency analysis.

Next, in step S6, the person detection unit 325 determines a person threshold value based on a maximum value of an intensity of the person signal or the like.

Next, in step S7, the person detection unit 325 detects the occupants M, and labels person signals corresponding to the occupants M in descending order of intensity.

Next, in step S8, the calculation unit 326 performs frequency analysis on each of the labeled person signals and calculates biological information.

Next, in step S9, the control unit 327 determines whether the vehicle is stopped, and the processing returns to step S1 if Yes, and proceeds to step S10 if No. In step S10, the processing unit 32 executes traveling processing.

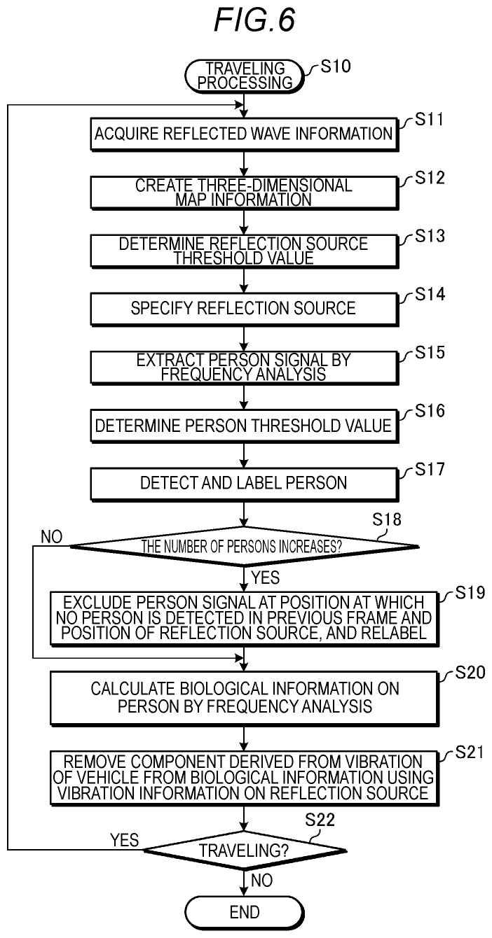

FIG. 6 is a flowchart showing details of processing of step S10 in FIG. 5. Steps S11 to S17 are the same as steps S1 to S7.

After step S17, in step S18, the person detection unit 325 determines whether the number of the detected occupants M increases compared to the number of the detected occupants M during stop, and the processing proceeds to step S19 if Yes and proceeds to step S20 if No.

In step S19, the person detection unit 325 excludes a person signal at a position at which no occupant M is detected in a previous frame (in a previous detection cycle) and which substantially matches the position of the reflection source, and performs re-labeling.

For example, it is detected in step S7 that there is a person in a driver seat and at a left end of a rear seat during stop. In step S17, it is detected that there is a person in the driver seat, a passenger seat, and at the left end of the rear seat during traveling, and the person in the driver seat, the person at the left end of the rear seat, and the person in the passenger seat are labeled in this order according to a magnitude of a signal intensity.

In step S18, it is recognized that the number of persons increases from two to three. Thereafter, in step S19, when the occupant M in the passenger seat is not detected in the previous frame and the position of the passenger seat substantially matches the position of the reflection source, a person signal of the passenger seat among the labeled person signals is excluded and relabeled.

In step S20, the calculation unit 326 performs frequency analysis on each of the labeled person signals and calculates biological information.

Next, in step S21, the calculation unit 326 removes a component derived from the vibration of the vehicle C from the biological information using the vibration information of the reflection source. By steps S20 and S21, for example, in the above example, the biological information is calculated for the person in the driver seat and the person at the left end of the rear seat, and the component derived from the vibration of the vehicle C is removed from the biological information.

Next, in step S22, the control unit 327 determines whether the vehicle is traveling, and the processing returns to step S11 if Yes, and ends if No.

Accordingly, according to the biological detection device 1 of the embodiment, when the vehicle C is traveling, a detected person who is not actually present can be identified by using the three-dimensional map information created by the FMCW method, the position of the reflection source, and the detection result of the motion of the object in the passenger compartment based on the Doppler shift of the reflected wave when the number of the detected persons increases compared to the number of detected persons when the vehicle is stopped, and thus the person in the passenger compartment can be detected with high accuracy.

The biological information can be detected with high accuracy by removing the component derived from the vibration of the vehicle C from the biological information of the person when the vehicle is traveling.

At least one of the pulse wave, the respiration, and the body motion of the person can be specifically detected as biological information with high accuracy. Therefore, for example, when a sudden disease occurs in the occupant M during traveling, the sudden disease can be detected with high accuracy if there is a predetermined change in the pulse wave, respiration, or body motion of the occupant M.

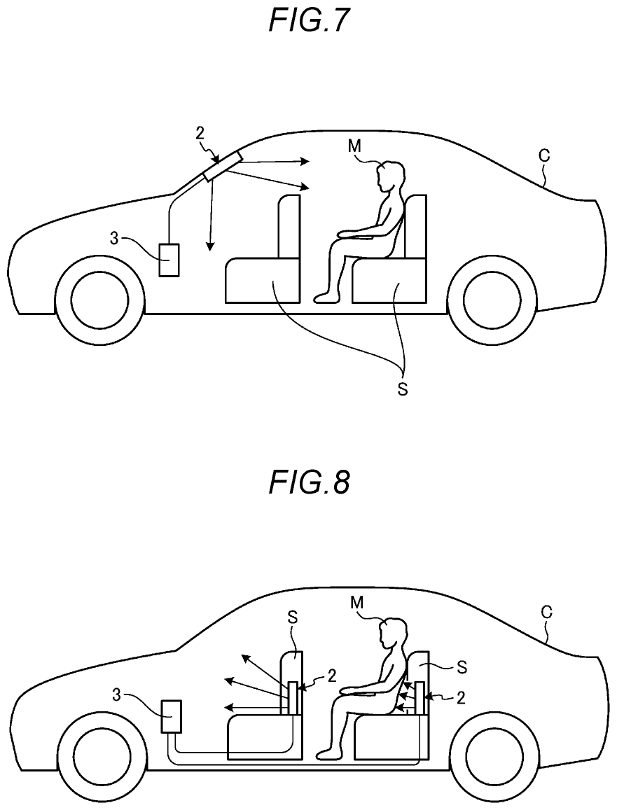

(Modification) FIG. 7 is a schematic diagram of the vehicle C according to a modification. In a passenger compartment of the vehicle C, the radio wave sensor 2 may be disposed at a front portion instead of a ceiling to detect an occupant and calculate biological information.

FIG. 8 is a schematic diagram of the vehicle C according to a modification. In a passenger compartment of the vehicle C, the radio wave sensor 2 may be disposed inside each seat S instead of the ceiling or the front portion to detect an occupant and calculate biological information.

The program executed by the controller 3 may be stored in a computer-readable storage medium such as a CD-ROM, a CD-R, a memory card, a digital versatile disk (DVD), or a flexible disk (FD) as a file in an installable format or an executable format and provided as a computer program product. The program may be stored in a computer connected to a network such as the Internet and provided by being downloaded via the network. The program may be provided or distributed via the network such as the Internet.

While the embodiment disclosed here has been described above, the embodiment has been presented by way of example only, and is not intended to limit the scope of the disclosure. Indeed, the novel embodiment may be embodied in a variety of other forms, and various omissions, substitutions and changes may be made without departing from the spirit of the disclosure. The embodiment and modifications thereof are included in the scope and gist of the disclosure, and are also included in the inventions described in the claims and equivalents thereof.

For example, in the radio wave sensor 2, instead of radiating a radio wave over a wide range and receiving a reflected wave by plural antennas, the reflected wave may be received by one antenna or plural antennas while changing a radiation direction of the radio wave. In either case, three-dimensional map information can be created based on information on the received reflected wave.

An optical sensor may be used instead of the radio wave sensor 2.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

A biological detection device according to an embodiment includes: a transmission unit configured to transmit a FMCW modulated transmission wave into a passenger compartment of a vehicle; a reception unit configured to receive a reflected wave generated by the transmission wave being reflected by an object in the passenger compartment; a creation unit configured to create three-dimensional map information on the passenger compartment based on the reflected wave; a specifying unit configured to specify a position of a reflection source, the reflection source being an object having a reflection intensity equal to or greater than a predetermined value with respect to the transmission wave, in the created three-dimensional map information when the vehicle in which at least one person is seated in a seat in the passenger compartment is stopped; an object detection unit configured to detect a motion of an object in the passenger compartment based on a Doppler shift of the reflected wave and output a detection result; and a person detection unit configured to detect, when the vehicle is stopped, a person based on the three-dimensional map information and the detection result in a predetermined detection cycle, and detect, when the vehicle is traveling, a person based on the three-dimensional map information and the detection result in a predetermined detection cycle, and determine that there is no person at a position at which no person is detected in an immediately preceding detection cycle and which substantially matches the position of the reflection source among positions at which persons are detected when the number of detected persons increases compared to the number of detected persons when the vehicle is stopped.

With such a configuration, when the vehicle is traveling, a detected person who is not actually present can be identified by using the three-dimensional map information created by a FMCW method, the position of the reflection source, and the detection result of the motion of the object in the passenger compartment based on the Doppler shift of the reflected wave when the number of the detected persons increases compared to the number of detected persons when the vehicle is stopped, and thus a person in the passenger compartment can be detected with high accuracy.

The biological detection device may further include: a calculation unit configured to, when the vehicle is traveling, calculate biological information with a motion related to a person based on the detection result, and remove a component derived from vibration of the vehicle from the biological information based on a motion of the reflection source that is close to the seat in which the person is seated and vibrates in correlation with the vibration when the seat vibrates.

With such a configuration, the biological information can be detected with high accuracy by removing the component derived from the vibration of the vehicle from the biological information of the person when the vehicle is traveling.

In the biological detection device, the biological information may be at least one of a pulse wave, respiration, and a body motion of the person. With such a configuration, at least one of the pulse wave, the respiration, and the body motion of the person can be specifically detected as biological information with high accuracy.

What is claimed is:

1. A biological detection device comprising:
a transmission unit configured to transmit a frequency modulated continuous wave modulated transmission wave into a passenger compartment of a vehicle;
a reception unit configured to receive a reflected wave generated by the transmission wave being reflected by an object in the passenger compartment;
a creation unit configured to create three-dimensional map information on the passenger compartment based on the reflected wave;
a specifying unit configured to specify a position of a reflection source, the reflection source being an object having a reflection intensity equal to or greater than a predetermined value with respect to the transmission wave, in the created three-dimensional map information when the vehicle in which at least one person is seated in a seat in the passenger compartment is stopped;
an object detection unit configured to detect a motion of an object in the passenger compartment based on a Doppler shift of the reflected wave and output a detection result; and
a person detection unit configured to detect, when the vehicle is stopped, a person based on the three-dimensional map information and the detection result in a predetermined detection cycle, and detect, when the vehicle is traveling, a person based on the three-dimensional map information and the detection result in a predetermined detection cycle, and determine that there is no person at a position at which no person is detected in an immediately preceding detection cycle and which substantially matches the position of the reflection source among positions at which persons are detected when the number of detected persons increases compared to the number of detected persons when the vehicle is stopped.

2. The biological detection device according to claim 1, further comprising:
a calculation unit configured to, when the vehicle is traveling, calculate biological information with a motion related to a person based on the detection result, and remove a component derived from vibration of the vehicle from the biological information based on a motion of the reflection source that is close to the seat in which the person is seated and vibrates in correlation with the vibration when the seat vibrates.

3. The biological detection device according to claim 2, wherein
the biological information is at least one of a pulse wave, respiration, and a body motion of the person.
